(19) United States
(12) Patent Application Publication
(10) Pub. No.: US 2023/0093543 A1
(43) Pub. Date: Mar. 23, 2023

(54) MULTIPLEX PCR METHOD FOR DETECTING MICROORGANISMS AND USE THEREOF

(71) Applicant: OCEAN DX, PESSAC (FR)

(72) Inventor: Franck TARENDEAU, LA BREDE (FR)

(21) Appl. No.: 17/795,989

(22) PCT Filed: Jan. 29, 2021
(86) PCT No.: PCT/EP2021/052115

§ 371 Date: Jul. 28, 2022

(30) Foreign Application Priority Data
Jan. 30, 2020 (FR) FR2000902

(51) Int. Cl. C12Q 1/689; C12Q 1/686

(52) U.S. Cl. CPC C12Q 1/689; C12Q 1/686; C12Q 2600/16

(57) ABSTRACT

The invention relates to a method for amplifying at least two distinct nucleic acid sequences present in a sample using a pair of primers (1) and a pair of primers (2). In particular, the first primer of the pair of primers (2) also comprises, in the (5′) position, the sequence of the first primer of the pair of primers (1), and the second primer of the pair of primers (2) also comprises, in the (5′) position, the sequence of the second primer of the pair of primers (1). The invention also relates to a kit and a detection kit capable of implementing said method and the uses thereof.

### FIELD OF THE INVENTION

The invention relates to the field of the detection and the amplification of at least one nucleic acid of interest, in particular in infection diagnosis. The invention thus relates to a method for amplifying at least two distinct nucleic acid sequences present in a sample, using a pair of primers 1 that amplify a first nucleic acid sequence (A), and at least one pair of hybrid or chimeric primers 2 that amplify a second nucleic acid sequence (B). In particular, the first primer, such as a sense primer of the pair of primers 2, further comprises, at 5′, the sequence of the first primer of the pair of primers 1, and the second primer of the pair of primers 2 further comprises, at 5′, the sequence of the first or of the second primer of the pair of primers 1. The invention also relates to the primers, a kit capable of implementing said method, and the uses thereof.

### BACKGROUND

In recent years, infection diagnosis has undergone significant development, in particular by the use of methods of molecular biology, such as genomic amplification or Polymerase Chain Reaction (PCR). The PCR is increasingly replacing the conventional serological and microbiological methods, such as the cultivation of bacteria or the ELISA tests. PCR has a number of advantages; the test is quick, sensitive, and specific. Despite still being relatively costly, and thus reserved for difficult analyses or for particular analysis laboratories, the specificity and the sensitivity of the diagnosis by PCR will eventually replace the conventional analyses which are implemented in the first instance.

The standard PCR makes it possible to amplify a nucleic acid of interest, from a specified pair of primers. In the case of infection diagnostics, for example, in the case of a suspected E. Coli infection, a pair of primers allowing for the amplification of a nucleic acid specific to E. Coli may be used. When the origin of the infection is not known, it is necessary to use pairs of primers that are specific to each of the pathogens which are intended to be detected. In a PCR approach referred to as “simplex”, a single pair of primers is used, and a PCR reaction, with respect to the number of viruses, bacteria or yeasts that are intended to be identified as responsible for the infection, has to be reproduced as many times as required. In a PCR approach referred to as “multiplex”, all the pairs of primers are added in one single reaction, and all the pathogens sought will be identified at the same time.

Multiplex PCR is a PCR that makes it possible to simultaneously identify a plurality of different nucleic acid sequences from the same biological sample. It makes it possible to perform a plurality of targeted amplifications in one and the same reaction. The multiplex PCR is therefore particularly suitable in infection diagnostics. During the infection diagnostics, the different pairs of primers are used for targeting a plurality of bacteria, viruses or yeasts during the same PCR reaction in the presence of the target biological sample.

However, the primer pairs used in multiplex PCR need to be significantly optimized in order that all the primers can work at the same temperature during the PCR, making it possible in particular to prevent the phenomenon of cross reactions.

By way of example, the multiplex PCR is capable of revealing at least one pathogen in the case of community-acquired pneumonia in 77% of hospitalized patients, compared with 39% by culture (N J Gadsby C D Russell M P McHugh Comprehensive molecular testing for respiratory pathogens in community-acquired pneumonia. Olin Infect Dis 2016), In the case of diarrhea, it makes it possible to detect up to three times more pathogens in the stools than the traditional methods (O R Stensvold H V Nielsen Comparison of microscopy and PCR for detection of intestinal parasites in Danish patients supports an incentive for molecular screening platforms. J Olin Microbiol 2012). Thus, one single text can demonstrate the presence, in the sample, of a plurality of infectious agents, in less than two hours. Today, panels are already proposed to clinicians for meningitis, sexually transmitted diseases, respiratory infections, and infectious enterocolitis.

Nonetheless, the multiplex PCR is limited to the detection of a predefined quantity of pathogens, of which the primer pairs targeting at least one fragment of a specific gene of said pathogens are included in the panel. In order to extend the detection panel, the detection kit may comprise a very large number of primers, thus making it possible to detect a large quantity of microorganisms. Thus, the more it is desirable to increase the number of pathogens targeted in a panel, the more there is a tendency to increase the number of pairs of primers. In a general manner, a pathogen is detected by a pair of primers, the amplicon of which is specific to said pathogen.

However, a high concentration of different primers in one single test brings about a deleterious contradictory effect. Thus, a competition phenomenon among the primers emerges, resulting in an inhibition of primers. Moreover, the non-specific amplifications are much more likely on account of the plurality of primers used. Consequently, multiplexing that is too high leads to a very poor detection sensitivity.

It is also known, from the work by I. Afonina et al. in 2007 (Primers with 5′ flaps improve real-time PCR), that the addition, at 5′, of a sequence that is not complementary to the target of the primer improves the effectiveness of PCR amplifications. However, these sequence additions to not address the problem of non-specific amplification or of loss of sensitivity, associated with the high concentrations of primers typically used during multiplex PCR.

The invention aims to solve the problems and overcome the disadvantages of the prior art, and in particular to prevent or limit the deterioration of the sensitivity of the multiplex PCR, which may result in lack of amplification or poor amplification of targeted nucleic acids, on account of the competition phenomenon between the primers when they are present in the same test, in quantities that are too large, as well as to suppress or limit the deterioration of the sensitivity of the multiplex PCR associated with non-specific amplifications which consume the amplification reagents in an undesired manner.

### INVENTION SUMMARY

Thus, according to a first aspect, the invention relates to a method for amplifying at least two distinct nucleic acid sequences of interest, present in a sample, using a pair of primers 1 capable of amplifying a first nucleic acid sequence of interest (A), and at least one pair of hybrid primers 2 capable of amplifying a second nucleic acid sequence of interest (B), said method comprising the following steps:
a. contacting the sample with the pair of primers 1 capable of amplifying the first nucleic acid sequence (A) and the pair of primers 2 capable of amplifying the second nucleic acid sequence (B),
   the pair of primers 1 being formed of a first primer and a second primer which are capable of amplifying a nucleic acid corresponding to the first nucleic acid sequence (A),
   the pair of primers 2 being formed of a first primer and a second primer which are capable of amplifying a nucleic acid corresponding to the second nucleic acid sequence (B),
      b. adding selected reagents, and conditions allowing for PCR amplification, and
      c. amplifying the first and second nucleic acid sequences (A and B), by performing an amplification reaction based on a specific transcription of the nucleic acid sequence (A) and of the nucleic acid sequence (B) present in the sample, characterized in that the sequence of the first primer of the pair of primers 2 further comprises, at 5′, the sequence of the first primer of the pair of primers 1, and the sequence of the second primer of the pair of primers 2 further comprises, at 5′, the sequence of the second primer of the pair of primers 1.

According to a variant, the sequence of the first primer of the pair of primers 2 (referred to as chimeric or hybrid) further comprises, at 5′, the sequence of the first or of the second primer of the pair of primers 1, and the sequence of the second primer of the pair of primers 2 further comprises, at 5′, the sequence of the first or of the second primer of the pair of primers 1.

The concentration of the pair of primers 2 is preferably less than or equal to that of the pair of primers 1, more preferably less.

The method according to the invention may also comprise a third pair of primers 3, a fourth pair of primers 4, etc., each comprising a first primer, the sequence of which further comprises, at 5′, the sequence of the first or of the second primer of the pair of primers 1, and a second primer, the sequence of which further comprises, at 5′, the sequence of the first or of the second primer of the pair of primers 1.

According to another variant, the second primer targeting the first nucleic acid sequence (A) can in turn also contain, in the region 5′ thereof, the sequence of the first primer targeting this same nucleic acid (A).

For example, the first oligonucleotide of the pair of primers 1, 2, 3 or 4 may be a sense or an antisense primer, and the second oligonucleotide of the pair of primers 1, 2, 3 or 4 may be an antisense or a sense primer.

Preferably, the method according to the invention is a multiplex PCR and makes it possible to perform differential quantitative amplifications, i.e. the targets are not amplified in the same way, leading to differences in quantities produced for the same number of cycles between the targets. Said PCR according to the invention is also referred to, in the remainder of the description, as Relay-PCR or PCR according to the invention.

The PCR according to the invention in particular makes it possible to detect and identify microorganisms such as bacteria, viruses, yeasts, which are involved in infectious diseases. The diseases may be detected in a subject such as an animal or a human, or in a plant.

According to a second aspect, the invention furthermore relates to the primers and kits comprising specific primers, a detection kit capable of implementing the method of the invention, and the use thereof for detecting the microorganisms present in the case of infectious diseases.

The invention is described in detail here with reference to the figures described in the following. The figures are merely illustrative, of some embodiments of the invention, and are not limiting.

### BRIEF DESCRIPTION OF THE DRAWINGS

FIG. 1 shows the principle of the Relay-PCR according to the invention.

FIG. 2A shows a standard Duplex-PCR test (multiplex having 2 targets), comprising a pair of primers 1 targeting Staphylococcus aureus, and a pair of primers 2 targeting Escherichia coli. The ratio of pair of primers 1:pair of primers 2 is 1:1. In black: the amplification curves targeting a specific gene of Escherichia coli. In gray: the amplification curves targeting a specific gene of Staphylococcus aureus.

FIG. 2B shows a Relay-PCR test according to the invention, comprising a pair of primers 1 targeting Staphylococcus aureus, and a pair of primers 2 targeting Escherichia coli. The ratio of pair of primers 1:pair of primers 2 is 10:1. In black: the amplification curves targeting a specific gene of Escherichia coli. In gray: the amplification curves targeting a specific gene of Staphylococcus aureus.

FIG. 2C shows a Relay-PCR test according to the invention, comprising a pair of primers 1 targeting Staphylococcus aureus, and a pair of primers 2 targeting Escherichia coli. The ratio of pair of primers 1:pair of primers 2 is 1:1. In black: the amplification curves targeting a specific gene of Escherichia coli. In gray: the amplification curves targeting a specific gene of Staphylococcus aureus.

FIG. 3A shows a standard Duplex-PCR test, comprising a pair of primers 1 targeting Klebsiella pneumoniae, and a pair of primers 2 targeting Staphylococcus aureus. The ratio of pair of primers 1:pair of primers 2 is 1:1. In black: the amplification curves targeting a specific gene of Klebsiella pneumoniae. In gray: the amplification curves targeting a specific gene of Staphylococcus aureus.

FIG. 3B shows a Relay-PCR test according to the invention, comprising a pair of primers 1 targeting Klebsiella pneumoniae, and a pair of primers 2 targeting Staphylococcus aureus. The ratio of pair of primers 1:pair of primers 2 is 10:1. In black: the amplification curves targeting a specific gene of Klebsiella pneumoniae. In gray: the amplification curves targeting a specific gene of Staphylococcus aureus.

FIG. 3C shows a Relay-PCR test according to the invention, comprising a pair of primers 1 targeting Klebsiella pneumoniae, and a pair of primers 2 targeting Staphylococcus aureus, The ratio of pair of primers 1:pair of primers 2 is 1:1. In black: the amplification curves targeting a specific gene of Klebsiella pneumoniae. In gray: the amplification curves targeting a specific gene of Staphylococcus aureus.

FIG. 4 shows the structure of a pair of primers used in standard PCR capable of amplifying a target sequence, for example A, comprising a sense primer of sense sequence FW1 and an antisense primer of sequence RT1; and the structure of a pair of hybrid or chimeric primers according to the invention used in Relay-PCT capable of amplifying a target sequence, for example B. The pair of hybrid or chimeric primers comprises a sense primer of sequence FW1+FW2 and an antisense primer of sequence RT1+RT2, The sequence FW2 is capable of hybridizing with a region of the target sequence B, the sequence FW1 corresponds to the sequence of the sense primer of the pair of primers 1 which is present in a higher concentration in the mixture, and said sequences FW1 and FW2 are adjacent so as to form a single sense primer of a length of between 12 and 140 nucleotides. The pair of hybrid or chimeric primers also comprises an antisense primer of sequence RT1+RT2, the sequence RT2 is complementary to a region of the target sequence B, the sequence RT1 corresponds to the sequence of the antisense primer of the pair of primers 1 which is present in a higher concentration in the mixture, said sequences FW1 and FW2 are adjacent so as to form a single primer of a length of between 12 and 140 nucleotides. With effect from the second PCR cycle, the pair of primers 1 present in a higher concentration in the mixture is capable of amplifying the amplicon generated by the pair of primers 2, during the first cycle, and thus of also amplifying the target sequence B. Thus, the pair of primers 1 is capable of amplifying the target sequence A but also the target sequence B from the second PCR cycle.

FIG. 5 shows the sequence of the bacterial gene 16S. Said sequence comprises: 9 variable regions that are specific to each species, and 10 conserved regions between the species, adjacent to said variable regions. The variable regions are numbered V1 to V9. The conserved regions make it possible to define, depending on the knowledge of a person skilled in the art, the nucleotide sequence of the sense and antisense primers capable of hybridizing on these conserved regions surrounding at least one variable region of interest (for example V1 to V2).

FIG. 6 is an agarose gel representing the results of PCR according to the standard Duplex method (multiplex having 2 targets) or according to the Duplex Relay method of the present invention. Under all conditions, 100 copies of the genome of Kiebsiella pneumoniae were added. Line 1: amplicon of the region 5′ of the gene 16S (amplicon covering the variable zones V1 to V2) obtained using a high concentration of primers targeting conserved zones adjacent to said variable zones (control PCR). 2: amplicon of the central region of the gene 16S (amplicon covering the variable zones V4 to V6) obtained using a high concentration of primers targeting conserved zones adjacent to said variable zones (control PCR). 3: amplicon in standard multiplex PCR of region 5′ (V1-V2) and the central region (V4-V6) of the gene 16S, using two pairs of primers targeting each variable zone, respectively, the primers being in high concentrations. 4: amplicon of the 2 regions of the gene 16S of the method of the present invention in Duplex Relay, where the first pair of primers targets the region 5′ (V1-V2) and the 2nd pair of hybrid or chimeric primers comprises a sequence that is capable of targeting the central region (V4-V6), but also the sequence of the first pair of primers, said primers are used at high (first pair of primers) and low (second pair of primers) concentrations, respectively. The arrows indicate the amplification or the lack of amplification of the amplicon of interest (in this case the region V1-V2 of the gene 16S).

FIG. 7 is an agarose gel representing the results of PCR according to the standard Triplex method (multiplex having 3 targets) or according to the method of the present invention (Triplex Relay). Under all conditions, 100 copies of the genome of Kiebsiella pneumoniae were added. 1: amplicons obtained using standard Triplex-PCR, the primers being 1 mM each. 2: amplicons obtained using Triplex Relay PCR, where the chimeric or hybrid primers were used at a low concentration of 0.04 mM. The amplicons V4-V6 and V7-V9 of the Triplex Relay are slightly larger than their equivalent in standard Triplex, on account of the length of the chimeric primers, which increase in size after polymerization.

FIG. 8 is an agarose gel representing the results of PCR according to the multiplex Relay-PCR method comprising one single primer in a high concentration. Under all conditions, 100 copies of the genome of Klebsiella pneumoniae were added. 1: amplicons obtained by means of Duplex Relay PCR, comprising a pair of primers 1 in a high concentration, targeting the region V1-V2 of the gene 16S, and a pair of hybrid or chimeric primers 2 in a low concentration, targeting the region V7-V9 of the gene 16S. 2: amplicons obtained by means of Duplex Relay PCR, comprising a pair of primers 1 targeting the region V1-V2 of the gene 16S, made up of a sense primer in a high concentration, and a hybrid or chimeric antisense primer in a low concentration, comprising, at 5′ the sense primer sequence, and a low concentration of a pair of hybrid or chimeric primers 2 targeting the region V7-V9 of the gene 16S. Thus, the antisense primer of the first pair, as well as the sense and antisense primers of the second pair, are hybrid or chimeric primers. Only the sense primer of the pair of primers 1 is present in a high concentration.

### DETAILED DESCRIPTION

### Definitions

Within the meaning of the invention, “patient” means a subject which may be a human being or an animal, preferably a human being or a mammal. The subject is preferably a human patient, whatever their age or gender. Newborns, infants and children are also included.

Within the meaning of the invention, “diagnosis” means a test which aims to detect and identify one or more pathology/pathologies, in particular to detect and identify infectious diseases in a subject or a plant, in order to be able to administer to the subject or to the plant a treatment that corresponds to the pathology.

Within the meaning of the invention, “pair of primers” or “pair of oligonucleotides” means two oligonucleotides which make it possible to amplify a target sequence within the context of a PCR.

Within the meaning of the invention, “primer” or “oligonucleotide” means a short segment of nucleic acids, of from one to a few tens of nucleotides. Said primers are capable of hybridizing with a complementary sequence. Within the context of the invention, they are capable of hybridizing with a fragment of the target sequence. The target sequence may be a nucleic acid sequence (DNA or RNA).

Within the meaning of the invention, “sequence FW1” means the sequence of an oligonucleotide of from one to several tens of nucleotides, in particular the sequence of a sense primer capable of hybridizing with the complementary sequence of the sequence of interest.

Within the meaning of the invention, “sequence RT1” means the sequence of an oligonucleotide of from one to several tens of nucleotides, in particular the sequence of an antisense primer capable of hybridizing with the complementary sequence of the sequence of interest.

Within the meaning of the invention, “pair of primers capable of amplifying a nucleic acid sequence of interest” means a pair of primers comprising a sense primer and an antisense primer capable of amplifying a specified sequence of interest, by means of PCR. By way of example, the nucleic sequence of interest may be a sequence present in the variable part of the gene 16S of a bacterium. Said primers are designed to target said sequence of interest in accordance with the general knowledge of a person skilled in the art.

Within the meaning of the invention, “amplification” means the amplification of the target sequence during a PCR reaction, and thus the increase in the number of copies of the target sequence. The primers hybridize with the previously denatured complementary sequence, according to the extension of the strands, by the action of the polymerase. The reaction proceeds with a plurality of successive cycles, making it possible to increase the number of copies of the amplicon.

Within the meaning of the invention, “amplicon” means a nucleic acid fragment that corresponds to the sequence that is amplified during the PRC and is delimited by the two primers used. The amplicon is thus a copy of the target sequence.

Within the meaning of the invention, “hybrid primer” or “chimeric primer” means a primer comprising a nucleic acid sequence that is capable of hybridizing with a complementary strand of the target sequence and, at 5′, a nucleic acid sequence corresponding to the sequence of a primer belonging to another primer pair, for example the sequence of a sense or antisense primer of another primer pair. Within the context of the invention, the pair of primers 2, 3 and 4 is formed of hybrid or chimeric primers. According to a variant of the invention, the pair of primers 1 comprises one “standard” primer and one hybrid or chimeric primer. For example, the first oligonucleotide of the pair of primers 2 comprises a sequence that is capable of hybridizing with a specific nucleic acid sequence (target B) and, at 5′, a nucleic acid sequence corresponding to the sequence of the first oligonucleotide of the pair of primers 1, which is capable of hybridizing with another specific nucleic acid sequence (target A).

Within the meaning of the invention, “concentration ratio” means the ratio of the concentration of the pair of primers 1 with respect to a second pair of primers, such as the pair of primers 2. For example, a ratio of 1:1 corresponds to an identical concentration of the first pair and of the second pair, and a ratio of 10:1 corresponds to a concentration of the first pair that is 10 times greater, compared with the second.

### DETAILED DESCRIPTION OF THE INVENTION

The invention thus relates to a new PCR method, referred to as multiplex. “Standard” multiplex PCRs are known which make it possible to identify a very large number of target sequences. Standard multiplex PCRs of this kind require the presence of a high concentration of each pair of primers targeting a particular nucleic acid. Thus, in standard multiplex PCR, each pair of primers is present at the same high concentration during the multiplex PCR reaction. Above at least 4 or 5 different pairs of primers, the reaction conditions begin to deteriorate. Furthermore, the emergence of a competition phenomenon among the primers results in an inhibition of specific hybridizations of primers. Moreover, the plurality of primers used may result in non-specific amplifications. This results in the standard multiplex PCRs having a very poor detection sensitivity.

The invention relates to a new multiplex PCR method, referred to as multiplex Relay-PCR (or Relay-PCR). In the case of multiplex Relay-PCR, i.e. the method according to the invention, one single pair of primers or one single primer is used in a high concentration; all the other primers are of a very low concentration. By virtue of the invention, it is possible to retain very high sensitivity for all the nucleic acids targeted.

It is also possible to promote the amplification of some nucleic acids over others (quantitative amplification asymmetry or differential quantitative amplification), in particular the nucleic acids of the target A, over the others. Said nucleic acids that are amplified preferentially will thus be present in a larger quantity. In diagnostics, favoring some nucleic acids has a medical advantage on account of the fact that some nucleic acids provide more information than others.

Thus, the invention relates to a method for amplifying at least two distinct nucleic acid sequences of interest, present in a sample, using a pair of primers 1 capable of amplifying a first nucleic acid sequence of interest (A), and at least one pair of primers 2 capable of amplifying a second nucleic acid sequence of interest (B), said method comprising the following steps:
a. contacting the sample with the pair of primers 1 capable of amplifying the first nucleic acid sequence (A) and the pair of primers 2 capable of amplifying the second nucleic acid sequence (B),
   i. the pair of primers 1 being formed of a first primer and a second primer which are capable of amplifying a nucleic acid corresponding to the first nucleic acid sequence (A),
   ii. the pair of primers 2 being formed of a first primer and a second primer which are capable of amplifying a nucleic acid corresponding to the second nucleic acid sequence (B),
b. adding selected reagents, and conditions allowing for PCR amplification, and
c. amplifying the first and second nucleic acid sequence (A and B), characterized in that the sequence of the first primer of the pair of primers 2 further comprises, at 5′, the sequence of the first primer of the pair of primers 1, and the sequence of the second primer of the pair of primers 2 further comprises, at 5′, the sequence of the second primer of the pair of primers 1.

During step c., the amplification of the first and second nucleic acid sequence (A and B) is performed by way of an amplification reaction based on polymerization (elongation) of the nucleic acid sequence (A) and of the nucleic acid sequence (B) present in the sample.

According to a variant, the sequence of the first primer of the pair of primers 2 further comprises, at 5′, the sequence of the first primer or of the second primer of the pair of primers 1, and the sequence of the second primer of the pair of primers 2 further comprises, at 5′, the sequence of the first or of the second primer of the pair of primers 1.

Preferably, the sequence of the first primer of the pair of primers 2 and the sequence of the first or of the second primer of the pair of primers 1 are adjacent, and the sequence of the second primer of the pair of primers 2 and the sequence of the first or of the second primer of the pair of primers 1 are adjacent. According to a variant, said sequences are not adjacent, but are spaced apart by a few nucleotides, by means of a spacer (or “linker”) of a size of between 1 and 12 nucleotides.

According to another variant, the method according to the invention is characterized in that:
the sequence of the second primer of the pair of primers 1 further comprises, at 5′, the sequence of the first primer of said pair of primers 1, said sequences are adjacent, and
the sequence of the first primer of the pair of primers 2 further comprises, at 5′, the sequence of the first primer of the pair of primers 1, and the sequence of the second primer of the pair of primers 2 further comprises, at 5′, the sequence of the first primer of the pair of primers 1, said sequences are adjacent.

According to another variant, the sequence of the second primer of the pair of primers 1 further comprises, at 5′, the sequence of the first primer of said pair of primers 1, and/or the sequence of the first primer of the pair of primers 2 further comprises, at 5′, the sequence of the first primer of the pair of primers 1, and the sequence of the second primer of the pair of primers 2 further comprises, at 5′, the sequence of the first primer of the pair of primers 1, said sequences are spaced apart by a few nucleotides, by means of a spacer (or “linker”) of a size of between 3 and 12 nucleotides.

According to a preferred embodiment of the invention, the method uses:
at least one pair of primers 1 capable of amplifying a target sequence A, comprising:
   a sense primer of sequence FW1 that is complementary to a fragment of the target sequence A, said primer being of a size of between 6 and 70 nucleotides, and
   an antisense primer of sequence RT1 that is complementary to a fragment of the target sequence A, said primer being of a size of between 6 and 70 nucleotides, and
at least one pair of primers 2 capable of amplifying a target sequence B, comprising:
   a sense primer of sequence FW1 or RT1 and of sequence FW2, the sequence FW2 being complementary to a fragment of the target sequence B, said sequences FW1 or RT1 and FW2 being adjacent in order to form a single primer of a size of between 12 and 140 nucleotides, and
   an antisense primer of sequence FW1 or RT1 and of sequence RT2, the sequence RT2 being complementary to a fragment of the target sequence B, said sequences FW1 or RT1 and RT2 being adjacent in order to form a single primer of a size of between 12 and 140 nucleotides.

Thus, the invention also relates to a pair of hybrid or chimeric primers, comprising:
a sense primer (or first oligonucleotide) of sequence FW1 or RT1 and of sequence FW2, the sequence FW2 is complementary to a fragment of the target sequence B, the sequence FW1 or RT1 corresponds, respectively, to the sequence of the sense or antisense primer of the pair of primers 1 present in a higher concentration in the mixture, said sequences FW1 or RT1 and FW2 are adjacent so as to form a single sense primer of a length of between 12 and 140 nucleotides, and
an antisense primer (or second oligonucleotide) of sequence FW1 or RT1 and of sequence RT2, the sequence RT2 is complementary to a fragment of the target sequence B, the sequence RT1 or FW1 corresponds, respectively, to the sequence of the antisense or sense primer of the pair of primers 1 present in an equal or higher concentration in the mixture, said sequences FW1 or RT1 and FW2 are adjacent so as to form a single sense primer of a length of between 12 and 140 nucleotides.

According to a variant, the invention also relates to a pair of hybrid or chimeric primers, comprising:
a sense or antisense primer (or first oligonucleotide) of sequence FW1 or RT1, the sequence FW1 or RT1 is complementary to a fragment of the target sequence A, present in a higher concentration in the mixture, said sequences are of a length of between 6 and 70 nucleotides, and
a hybrid or chimeric sense or antisense primer (or second oligonucleotide) of sequence FW1 and of sequence RT1, or of sequence RT1 and of sequence FW1, said primers being present in the mixture in a lower concentration compared with the first oligonucleotide of sequence FW1 or RT1, said sequences FW1 and RT1 or RT1 and FW1 are adjacent so as to form a single primer of a length of between 12 and 140 nucleotides.

During Duplex-Relay PCR, according to the present invention, the PCR reaction takes place in a mixture comprising a pair of primers 1 capable of hybridizing and amplifying a first nucleic acid sequence (A), and a pair of primers 2 capable of hybridizing and amplifying a second nucleic acid sequence (B), as is the case for a standard Duplex-PCR However, the Duplex-Relay PCR differs from standard Duplex-PCR in the use of hybrid or chimeric primers for the second pair of primers, and optionally for a sense or antisense primer of the first pair of primers.

Thus, with effect from the second PCR cycle, the pair of primers 1 is capable of also amplifying the amplicon generated by the pair of primers 2, and thus the target sequence B.

The Duplex Relay PCR in particular comprises contacting the sample with the reaction mixture comprising the pair of primers 1 capable of amplifying the nucleic acid sequence (A) and the pair of primers 2 capable of amplifying the second nucleic acid sequence (B).

The pair of primers 1 is made up of a first sense primer (for example of sequence FW1) and a second antisense primer (for example of sequence RT1) which are capable of each hybridizing to the complementary sequence of one of the strands of the nucleic acid (A), and of amplifying the sequence corresponding to the complementary sequence of the strand between the two primers. Said complementary sequence is the target sequence (A).

The pair of primers 2 is made up of a first sense primer (for example of sequence FW1-FW2) and a second antisense primer (for example of sequence RT1-RT2) which are capable of each hybridizing to the complementary sequence of one of the strands of the nucleic acid (B), and of amplifying the sequence corresponding to the complementary sequence of the strand between the two primers. Said complementary sequence is the target sequence (B).

Furthermore, the reaction mixture comprises all the reagents and conditions necessary for performing PCR amplification. Said reagents can be initially present in the reaction mixture, or added following contacting of the sample. The required reagents include in particular dNTPs and polymerase, which a person skilled in the art would be capable of selecting, depending on the PCR and the conditions of the reaction. The polymerase is preferably polymerase of Thermophilus aquaticus, or the “Klenow” fragment of said polymerase.

Once all the constituents are present in the reaction mixture, the amplification can begin.

The various steps of a PCR reaction are known to a person skilled in the art. A PCR cycle comprises a step of denaturation, which aims to separate the double strand in order to obtain two single strands, followed by the hybridization step. During this step, the primers will recognize the complementary sequence present on one of the single strands, previously denatured, and will then hybridize. This is followed by a step of elongation, which makes it possible for the polymerases to synthesize the strand complementary to the single strand. Said strand is synthesized from free dNTPs present in the reaction mixture. The duration of this step depends on the length of the sequence to be amplified.

The PCR reaction continues with a second cycle, third cycle, etc., which each comprise the same steps. Thus, at the end of the PCR, an amplification of the sequence contained between the primers is achieved. Said sequence corresponds to the amplicon.

Thus, the amplification of the first and second nucleic acid sequence is based on an amplification of the target nucleic acid sequence (A) and of the target nucleic acid sequence (B) present in the sample, preferably a simultaneous amplification of the target nucleic acid sequence (A) and of the target nucleic acid sequence (B) present in the sample.

The method according to the invention is characterized in that the first primer of the pair of primers 2 further comprises, at 5′, the sequence of the first primer of the pair of primers 1, and the second primer of the pair of primers 2 further comprises, at 5′, the sequence of the second primer of the pair of primers 1. The sequence of the first primer of the pair 2 and of the first primer of the pair 1 are adjacent; and the sequences of the second primer of the pair 2 and of the second primer of the pair 1 are also adjacent.

According to a variant, the second primer of the pair of primers 2 further comprises; at 5′, the sequence of the first or of the second primer of the pair of primers 1. The sequence of the first primer of the pair 2 and of the first primer of the pair 1 are adjacent; and the sequences of the first or of the second primer of the pair 2 and of the second primer of the pair 1 are also adjacent.

Preferably, the first primer of the pair of primers 2 comprises a nucleic acid sequence capable of hybridizing with the complementary sequence of the sequence to be amplified, such as the target sequence (B), and furthermore, at 5′, the sequence of the first or second primer of the pair of primers 1, and the second primer of the pair of primers 2 comprises a nucleic acid sequence capable of hybridizing with the complementary sequence of the sequence to be amplified, such as the target sequence (B), and furthermore, at 5′, the sequence of the first or second primer of the pair of primers 1. The nucleic acid sequence capable of hybridizing with the complementary sequence to be amplified, such as the target sequence (B), is different for the primer 1 and the primer 2. In particular, the primer 1 may be a sense primer, and the primer 2 may be an antisense primer.

Preferably, the first pair of primers is capable of amplifying the first sequence (A) and the second sequence (B), with effect from the second PCR cycle. Indeed, during the first PCR cycle, the first pair of primers is capable of amplifying the first sequence (A), and the second pair of primers is capable of amplifying the second sequence (B). At the end of the first PCR cycle, nucleic acid sequences are generated comprising the sequence of the first primer of the first pair and the complementary sequence of the sequence (A), nucleic acid sequences comprising the sequence of the second primer of the first pair and the complementary sequence of the sequence (A), nucleic acid sequences comprising the sequence of the first primer of the second pair and the complementary sequence of the sequence (B), and nucleic acid sequences comprising the sequence of the second primer of the second pair and the complementary sequence of the sequence (B).

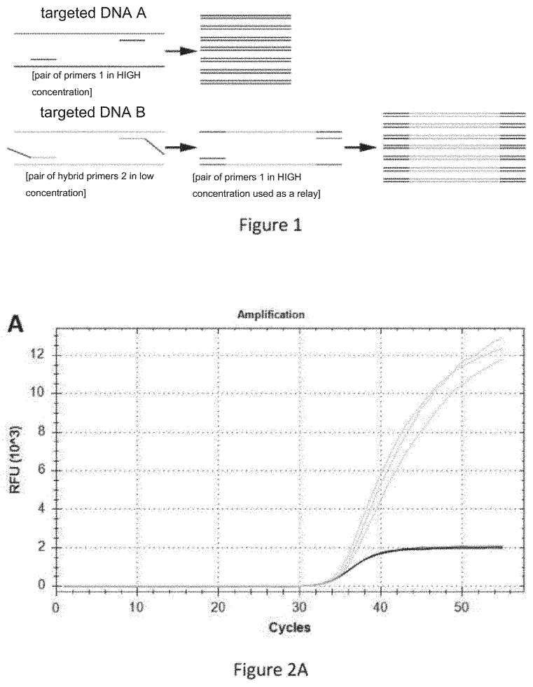

Indeed, the first primer of the second pair comprises the sequence of the first primer of the first pair and of a sequence capable of hybridizing with the sequence (B), and the second primer of the second pair comprises the sequence of the second primer of the first pair and of a sequence capable of hybridizing with the sequence (B). Thus, during the second cycle, the pair of primers 1 is capable of amplifying the nucleic acid sequences generated during the first cycle (FIG. 1).

According to one embodiment, the sequence of the first primer of the first pair is selected from SEQ ID NO: 1, SEQ ID NO: 3 or SEQ ID NO: 5, the sequence of the second primer of the first pair is selected from SEQ ID NO: 2, SEQ ID NO: 4 or SEQ ID NO: 6, the sequence of the first primer of the second pair is selected from SEQ ID NO: 7 or SEQ ID NO: 9, the sequence of the second primer of the second pair is selected from SEQ ID NO: 8 or SEQ ID NO: 10. Said sequences are listed in table 1, below.

TABLE 1A

| | | | |
|---|---|---|---|
| 1^{st} pair | 1^{st} primer | CGGAAGCAACGCGTAAACTC | SEQ ID No: 1 |
| | 2^{nd} primer | TGAGCGTCGCAGAACATTACA | SEQ ID No: 2 |
| 1^{st} pair | 1^{st} primer | TCGAAATTAAATGTTGTCGTGTCTTC | SEQ ID No: 3 |
| | 2^{nd} primer | CAATATTTTCATTTTTGACATGGAGAGAAACATC | SEQ ID No: 4 |
| 1^{st} pair | 1^{st} primer | GACGGGATATCTGACCAGTCGG | SEQ ID No: 5 |
| | 2^{nd} primer | CCGGGTTTTGCGTAATGATCTG | SEQ ID No: 6 |
| 2^{nd} pair | 1^{st} primer | TCGAAATTAAATGTTGTCGTGTCTTCCGGAAGCAA | SEQ ID No: 7 |
| | | CGCGTAAACTC | |
| | 2^{nd} primer | CAATATTTTCATTTTTGACATGGAGAGAAACATCT | SEQ ID No: 8 |
| | | GAGCGTCGCAGAACATTACA | |
| 2^{nd} pair | 1^{st} primer | GACGGGATATCTGACCAGTCGGTCGAAATTAAATG | SEQ ID No: 9 |
| | | TTGTCGTGTCTTC | |
| | 2^{nd} primer | CCGGGTTTTGCGTAATGATCTGCAATATTTTCATT | SEQ ID No: 10 |
| | | TTTGACATGGAGAGAAACATC | |

For purposes of diagnoses, it is of interest to target the variable regions of the gene 16S present in all the bacteria.

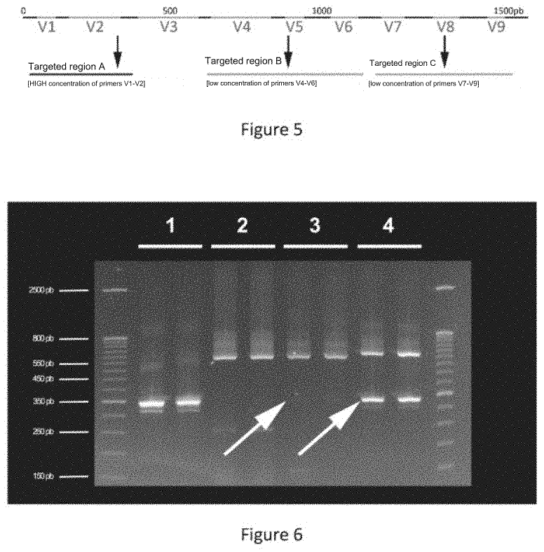

The gene 16S comprises conserved regions and variable regions, in particular 9 variable regions and 10 conserved regions (cf. FIG. 5). The variable regions are numbered V1 to V9 and are all surrounded by conserved regions. On the basis of his general knowledge, a person skilled in the art is entirely capable of determining said regions on the gene 16S. Tremblay et al. 2015 (Frontiers in Microbiology) describes, for example, one of these numerous methods for amplifying and sequencing said regions, in order to identify the bacteria present in a sample.

Thus, according to a preferred embodiment, the sequences of interest correspond to variable sequences of a single gene or of at least two separate genes, more preferably the sequences of interest correspond to the variable sequences of the gene 16S, and said primers are capable of hybridizing with at least a dozen nucleotides of a conserved sequence of the gene 16S. Preferably, the sense primer can hybridize with at least a dozen nucleotides of a conserved sequence of the gene 16S at position 5′ of a variable sequence of said gene 16S, and the antisense primer can hybridize with at least a dozen nucleotides of a conserved sequence of the gene 16S at position 3′ of a variable sequence of said gene 16S.

Within the context of the present invention, the inventors have designed primers which make it possible to hybridize specifically on the conserved regions adjacent to the variable regions of interest, such as V1-V2, V3, V4-V6 and V7-V9. Thus, the amplicon generated corresponds to the sequences of the variable regions V1-V2 or V3 or V4-V6 or V7-V9, the sequence of which is specific to one pathogen.

According to a preferred embodiment, the sequence of the first primer (sense primer) of the first pair is selected from SEQ ID NO: 14, SEQ ID NO: 15, SEQ ID NO: 18, SEQ ID NO: 20, SEQ ID NO: 21 and SEQ ID NO: 24; the sequence of the second primer (antisense primer) of the first pair is selected from SEQ ID NO: 16, SEQ ID NO: 17, SEQ ID NO: 19, SEQ ID NO: 22, SEQ ID NO: 23, SEQ ID NO: 25, SEQ ID NO: 26 and SEQ ID NO: 27.

Preferably, the sequence of the first primer (sense primer) of the first pair SEQ ID NO: 14 or SEQ ID NO: 15, and the sequence of the second primer (antisense primer) of the first pair is selected from SEQ ID NO: 16, SEQ ID NO: 17, SEQ ID NO: 26 and SEQ ID NO: 27.

The sequence of the first primer (sense primer) of the second pair is selected from SEQ ID NO: 28, SEQ ID NO: 29, SEQ ID NO: 30, SEQ ID NO: S4 or SEQ ID NO: 35; the sequence of the second primer of the second pair is selected from SEQ ID NO: 31, SEQ ID NO: 32, SEQ ID NO: 33, SEQ ID NO: 36, SEQ ID NO: 37, or SEQ ID NO: 38. Said sequences are listed in table 1B, below.

TABLE 1B

| | | Variable region |
|---|---|---|
| Primer | | targeted, and |
| Sequences | Sequence (5′-3′) | orientation |
| SEQ ID No: 14 | AGAGTTTGATCCTGGCTCAG | V1sense |
| SEQ ID No: 15 | GCCTAACACATGCAAGT | V1sense |
| SEQ ID No: 16 | GCCTCCCGTAGGAG | V2antisense |
| SEQ ID No: 17 | TCTGGACCGTGTCTCAGT | V2antisense |
| SEQ ID No: 18 | CTCCTACGGGAGGC | V3sense |
| SEQ ID No: 19 | ACCGCGGCTGCT | V3antisense |
| SEQ ID No: 20 | CGTGCCAGCAGCCGCGGT | V4sense |
| SEQ ID No: 21 | CGGAATTACTGGGCGTAAA | V4sense |
| SEQ ID No: 22 | ACACGAGCTGACGAC | Veantisense |
| SEQ ID No: 23 | TCACGACACGAGCTGACGAC | V6antisense |
| SEQ ID No: 24 | AACGAGCGCAACCC | V7sense |
| SEQ ID No: 25 | TACGGTTACCTTGTTACGACTT | V9antisense |
| Hybrid or | | Variable regins |
| chimeric primer | | targeted, and |
| sequences | Sequence (5′-3′) | orientation |
| SEQ ID No: 26 | AGATTTGATCCTGGCTCAGTCTGGACCGTGTCTCAGT | V1sense- |
| | | V2antisense |
| SEQ ID No: 27 | GCCTAACACATGCAAGTTCTGGACCGTGTCTCAGT | V1sense- |
| | | V2antisense |
| SEQ ID No: 28 | AGAGTTTGATCCTGGCTCAGCGTGCCAGCAGCCGCGGT | V1sense- |
| | | V4sense |
| SEQ ID No: 29 | AGAGTTTGATCCTGGCTCAGCGGAATTACTGGGCGTAAA | V1sense- |
| | | V4sense |
| SEQ ID No: 30 | GCCTAACACATGCAAGTCGGAATTACTGGGCGTAAA | V1sense- |
| | | V4sense |
| SEQ ID No: 31 | AGAGTTTGATCCTGGCTCAGACACGAGCTGACGAC | V1sense- |
| | | V6antisense |
| SEQ ID No: 32 | GCCTAACACATGCAAGTACACGAGCTGACGAC | V1sense- |
| | | V6antisense |
| SEQ ID No: 33 | GCCTCCCGTAGGAGTCACGACACGAGCTGACGAC | V2antisense- |
| | | V6antisense |
| SEQ ID No: 34 | AGAGTTTGATCCTGGCTCAGAACGAGCGCAACCC | V1sense- |
| | | V7sense |
| SEQ ID No: 35 | TCTGGACCGTGTCTCAGTAACGAGCGCAACCC | V2antisense- |
| | | V7sense |
| SEQ ID No: 36 | GCCTCCCGTAGGAGTACGGTTACCTTGTTACGACTT | V2antisense- |
| | | V9antisense |
| SEQ ID No: 37 | AGAGTTTGATCCTGGCTCAGTACGGTTACCTTGTTACGACTT | V1sense- |
| | | V9antisense |
| SEQ ID No: 38 | GCCTAACACATGCAAGTTACGGTTACCTTGTTACGACTT | V1sense- |
| | | V9antisense |

The first pair of primers targeting the variable regions V1-V2 of the gene 16S is preferably made up:
of a sense primer having at least 70% sequence homology with SEQ ID NO: 14 or SEQ ID NO: 15, and
of an antisense primer having at least 70% sequence homology with SEQ ID NO: 16 or SEQ ID NO: 17 or SEQ ID NO: 26 or SEQ ID NO: 27.

The second pair of hybrid or chimeric primers targeting the variable regions V4 to V6 of the gene 16S is preferably made up:
of a sense primer having at least 70% sequence homology with SEQ ID NO: 28 or SEQ ID NO: 29 or SEQ ID NO: 30, and
of an antisense primer having at least 70% sequence homology with SEQ ID NO: 31 or SEQ ID NO: 32 or SEQ ID NO: 33.

The second pair or the third pair of primers targeting the variable regions V7 to V9 of the gene 16S is preferably made up:
of a sense primer having at least 70% sequence homology with SEQ ID NO: 34 or SEQ ID NO: 35, and
of an antisense primer having at least 70% sequence homology with SEQ ID NO: 36 or SEQ ID NO: 37 or SEQ ID NO: 38.

The sequence homology of 70% preferably relates, in particular, to the first ten nucleotides at position 3′ of said sequence.

More preferably, the first pair of primers targeting the variable regions V1-V2 of the gene 16S is made up:
of a sense primer of SEQ ID NO: 14 or SEQ ID NO: 15, and
of an antisense primer SEQ ID NO: 16 or SEQ ID NO: 17 or SEQ ID NO: 26 or SEQ ID NO: 27.

The second pair of hybrid or chimeric primers targeting the variable regions V4 to V6 of the gene 16S is preferably made up:
of a sense primer of SEQ ID NO: 28 or SEQ ID NO: 29 or SEQ ID NO: 30, and
of an antisense primer SEQ ID NO: 31 or SEQ ID NO: 32 or SEQ ID NO: 33.

The second pair or the third pair of primers targeting the variable regions V7 to V9 of the gene 16S is preferably made up:
of a sense primer of SEQ ID NO: 34 or SEQ ID NO: 35, and
of an antisense primer SEQ ID NO: 36 or SEQ ID NO: 37 or SEQ ID NO: 38.

During the method according to the invention, the concentration of the pair of primers 2 may be less than, greater than or equal to that of the pair of primers 1. It is thus possible to promote, or not, one pair of primers compared with another, and thus the amplification of one sequence of interest.

According to a particularly preferred embodiment of the invention, the concentration of the pair of primers 2 is less than or equal to that of the pair of primers 1, more preferably less. When the concentration of the pair of primers 2 is less than that of the pair of primers 1, the pair of primers 2 is disadvantaged with respect to the first pair. The amplicons generated by the pair of primers 1 will thus be present in a larger quantity. When sequencing is then performed, the amplicons of the target A will be over-represented, and will thus be sequenced preferentially. The sequence thereof will thus be obtained more quickly.

The method according to the invention makes it possible to generate a quantity of amplicons, by way of the pair of primers 1, that is at least 4 times greater than the amplicons generated by the pair of primers 2. The amplicons generated by the second pair will thus be present in a smaller quantity, and will be eliminated less quickly during sequencing.

The concentration ratio between the pair of primers 1 and the pair of primers 2 is preferably between 1:1 and 1000:1, even more preferably between 1:1 and 100:1. According to a particularly advantageous embodiment, the concentration ratio between the pair of primers 1 and the pair of primers 2 is 10:1, more preferably between 25:1. A ratio of this kind may for example correspond to a concentration of the pair of primers 1 of 1 mM, and a concentration of the pair of primers 2 of 0.1 mM or 0.04 mM.

According to another embodiment, the method according to the invention may furthermore comprise contacting the sample with a 3rd pair of primers capable of amplifying a 3rd nucleic acid sequence (C). The first primer of the pair of primers 3 comprises a nucleic acid sequence capable of hybridizing with the complementary sequence (C), and furthermore, at 5′, the sequence corresponding to the sequence of the first primer or of the second primer of the pair of primers 1. The second primer of the pair of primers 3 comprises a nucleic acid sequence capable of hybridizing with the complementary sequence (C), and furthermore, at 5′, the sequence corresponding to the sequence of the first or of the second primer of the pair of primers 1.

The method according to the invention thus comprises 3 pairs of different primers which make it possible to amplify 3 different target nucleic acid sequences, in particular a sequence A, a sequence B and a sequence C. Said method is a PCR referred to as Triplex Relay.

The sequences B and C are preferably amplified by hybrid or chimeric sense and antisense primers, each comprising the sequence of the sense primer of the pair of primers 1 present in a higher concentration, and the sequence of the sense or antisense primer of the pair of primers 1 present in a higher concentration.

Thus, the method according to the invention uses:
at least one pair of primers 1 capable of amplifying a target sequence A, comprising:
   a sense primer of sequence FW1 that is complementary to a fragment of the target sequence A, said primer being of a size of between 6 and 70 nucleotides, and
   an antisense primer of sequence RT1 that is complementary to a fragment of the target sequence A, said primer being of a size of between 6 and 70 nucleotides, and
at least one pair of hybrid or chimeric primers 2 capable of amplifying a target sequence B, comprising:
   a sense primer of sequence FW1 or RT1 and of sequence FW2, the sequence FW2 being complementary to a fragment of the target sequence B, said sequence FW1 or RT1 and FW2 being adjacent in order to form a single primer of a size of between 12 and 140 nucleotides, and
   an antisense primer of sequence FW1 or RT1 and of sequence RT2, the sequence RT2 being complementary to a fragment of the target sequence B, said sequence FW1 or RT1 and RT2 being adjacent in order to form a single primer of a size of between 12 and 140 nucleotides, and
at least one pair of primers 3 capable of amplifying a target sequence C, comprising:
   a sense primer of sequence FW1 and of sequence FW3, the sequence FW3 being complementary to a fragment of the target sequence C, said sequence FW1 and FW3 being adjacent in order to form a single primer of a size of between 12 and 140 nucleotides, and
   an antisense primer of sequence FW1 or RT1 and of sequence RT3, the sequence RT3 being complementary to a fragment of the target sequence C, said sequence FW1 or RT1 and RT3 being adjacent in order to form a single primer of a size of between 12 and 140 nucleotides.

During the method, the concentration of the pair of primers 3 is less than or greater than or equal to that of the pair of primers 1, and/or the concentration of the pair of primers 2 is less than or greater than or equal to that of the pair of primers 1. It is thus possible to promote, or not, one pair of primers compared with another. Preferably, the concentration of the pair of primers 3 is less than that of the pair of primers 1, and/or the concentration of the pair of primers 2 is less than that of the pair of primers 1.

Preferably, the concentration of the pair of primers 1 is greater than that of the second and/or of the third pair of primers, and the concentration of the pair of primers 2 is equal to that of the pair of primers 3.

According to one embodiment, the concentration of the pair of primers 1 is greater than that of the second and third pair of primers, and the concentration of the pair of primers 2 is greater than that of the pair of primers 3. Thus, the first pair is promoted in comparison with the second, and the second is also promoted in comparison with the third.

According to another embodiment, the method according to the invention may furthermore comprise contacting the sample with a fourth pair of primers, referred to as hybrid or chimeric, capable of amplifying a fourth nucleic acid sequence (D). The first primer of the pair of primers 4 comprises a nucleic acid sequence capable of hybridizing with the complementary sequence (D), and furthermore, at 5′, the sequence corresponding to the sequence of the first primer or of the second primer of the pair of primers 1. The second primer of the pair of primers 4 comprises a nucleic acid sequence capable of hybridizing with the complementary sequence (D), and furthermore, at 5′, the sequence corresponding to the sequence of the first primer or of the second primer of the pair of primers 1.

Preferably, the concentration of the fourth pair of primers is less than or equal to the concentration of the pair of primers 1, and the concentration of the second, third and fourth pair is identical.

According to another embodiment, the method according to the invention may furthermore comprise contacting the sample with an nth pair of primers capable of amplifying an nth nucleic acid sequence. The first primer of the pair of primers n comprises a nucleic acid sequence capable of hybridizing with the target complementary sequence, and furthermore, at 5′, the sequence corresponding to the sequence of the first primer or of the second primer of the pair of primers 1. The second primer of the pair of primers n comprises a nucleic acid sequence capable of hybridizing with the target complementary sequence, and furthermore, at 5′, the sequence corresponding to the sequence of the first primer or of the second primer of the pair of primers 1.

Preferably, the concentration of the nth pair of primers is less than or equal to the concentration of the pair of primers 1, and the concentration of the second, third, fourth pair and of the nth pair is identical.

Within the meaning of the invention, “n” means a natural number which is a positive number making it possible to enumerate objects, counting each as one. Thus, a whole number as a single successor, i.e. a whole number which is immediately greater than it, and the list of natural numbers is infinite.

Furthermore, the reaction medium making it possible to carry out the method according to the invention may comprise one or more specific probe(s) coupled to a fluorophore, the emission spectrum of which is different from the others. Thus, each product of the PCR reaction is measured in real time, by said specific probe(s).

Thus, according to one embodiment, the method according to the invention further comprises a step of quantification of the amplicons by using probes coupled to a fluorophore, preferably “TaqMan” probes.

Probes of this kind according to the invention are listed in table 2, below.

TABLE 2

| | | |
|---|---|---|
| Probe 1 (EC) | CGCGTCCGATCACCTGCGTC | SEQ ID No: 11 |
| Probe 2 (KP) | GTTGAGGTCAACAGTGCTGCGG | SEQ ID No: 12 |
| Probe 3 (SA) | TCGCGACATTCATTATGCCA | SEQ ID No: 13 |

According to another embodiment, the method according to the invention comprises a step of detection of amplicons, and thus microorganisms, by fluorescent probes, fluorescent marking, melting curve, nested PCR, quantitative PCR, reverse transcription PCR, or by DNA sequencing.

When the detection of the amplicons is performed by sequencing, the method further comprises a step of sequencing amplified nucleic acids or amplicons.

According to a particularly preferred embodiment, the method according to the invention makes it possible to detect and identify microorganisms (bacteria, yeasts, viruses, etc.) involved in at least one infectious disease, preferably bacteria.

The method according to the invention can also be used for diagnosis of genetic diseases, in oncology, and, in a general manner, in molecular biology.

According to another aspect, the invention relates to a kit comprising a pair of primers 1 comprising SEQ ID No: 1 and SEQ ID No: 2, or SEQ ID No: 3 and SEQ ID No: 4, or SEQ ID No: 5 and SEQ ID No: 6, and a pair of primers 2 comprising SEQ ID No: 7 and SEQ ID No: 8, or SEQ ID No: 9 and SEQ ID No: 10.

According to a preferred embodiment, the invention relates to a kit comprising:
a pair of primers 1 targeting the variable regions V1-V2 of the gene 16S made up:
   of a sense primer having at least 70% sequence homology with SEQ ID No: 14 or SEQ ID No: 15, and
   of an antisense primer having at least 70% sequence homology with SEQ ID No: 16 or SEQ ID No: 17 or SEQ ID No: 26 or SEQ ID No: 27,
a pair of primers 2 targeting the variable regions V4-V6 of the gene 16S made up:
   of a sense primer having at least 70% sequence homology with SEQ ID No: 28 or SEQ ID No: 29 or SEQ ID No: 30, and
   of an antisense primer having at least 70% sequence homology with SEQ ID No: 31 or SEQ ID No: 32 or SEQ ID No: 33.

According to another preferred embodiment, the invention relates to a kit comprising:
a pair of primers 1 targeting the variable regions V1-V2 of the gene 16S made up:
   of a sense primer having at least 70% sequence homology with SEQ ID No: 14 or SEQ ID No: 15, and
   of an antisense primer having at least 70% sequence homology with SEQ ID No: 16 or SEQ ID No: 17 or SEQ ID No: 26 or SEQ ID No: 27,
a pair of primers 2 targeting the variable regions V7-V9 of the gene 16S made up:
   of a sense primer having at least 70% sequence homology with SEQ ID No: 34 or SEQ ID No: 35, and
   of an antisense primer having at least 70% sequence homology with SEQ ID No: 36 or SEQ ID No: 37 or SEQ ID No: 38.

According to another embodiment, the invention relates to a kit comprising:
a pair of primers 1 targeting the variable regions V1-V2 of the gene 16S made up:
   of a sense primer having at least 70% sequence homology with SEQ ID No: 14 or SEQ ID No: 15, and
   of an antisense primer having at least 70% sequence homology with SEQ ID No: 16 or SEQ ID No: 17 or SEQ ID No: 26 or SEQ ID No: 27,
a pair of primers 2 targeting the variable regions V4-V6 of the gene 16S made up:
   of a sense primer having at least 70% sequence homology with SEQ ID No: 28 or SEQ ID No: 29 or SEQ ID No: 30, and
   of an antisense primer having at least 70% sequence homology with SEQ ID No: 31 or SEQ ID No: 32 or SEQ ID No: 33,
a pair of primers 3 targeting the variable regions V7-V9 of the gene 16S made up:
   of a sense primer having at least 70% sequence homology with SEQ ID No: 34 or SEQ ID No: 35, and
   of an antisense primer having at least 70% sequence homology with SEQ ID No: 36 or SEQ ID No: 37 or SEQ ID No: 38.

The kit according to any of the embodiments above preferably comprises primers of which the sequence homology of 70% relates, in particular, to the first ten nucleotides at position 3′ of said sequence.

According to a particularly preferred embodiment, the invention relates to a kit comprising:
a pair of primers 1 targeting the variable regions V1-V2 of the gene 16S made up:
   of a sense primer of SEQ ID No: 14 or SEQ ID No: 15, and
   of an antisense primer SEQ ID No: 16 or SEQ ID No: 17 or SEQ ID No: 26 or SEQ ID No: 27,
a pair of primers 2 targeting the variable regions V4-V6 of the gene 16S made up:
   of a sense primer of SEQ ID No: 28 or SEQ ID No: 29 or SEQ ID No: 30, and
   of an antisense primer SEQ ID No: 31 or SEQ ID No: 32 or SEQ ID No: 33,

According to a particularly preferred embodiment, the invention relates to a kit comprising:
a pair of primers 1 targeting the variable regions V1-V2 of the gene 16S made up:
   of a sense primer of SEQ ID No: 14 or SEQ ID No: 15, and
   of an antisense primer SEQ ID No: 16 or SEQ ID No: 17 or SEQ ID No: 26 or SEQ ID No: 27,
a pair of primers 2 targeting the variable regions V7-V9 of the gene 16S made up:
   of a sense primer of SEQ ID No: 34 or SEQ ID No: 35, and
   of an antisense primer SEQ ID No: 36 or SEQ ID No: 37 or SEQ ID No: 38.

According to another particularly preferred embodiment, the invention relates to a kit comprising:
a pair of primers 1 targeting the variable regions V1-V2 of the gene 16S made up:
   of a sense primer of SEQ ID No: 14 or SEQ ID No: 15, and
   of an antisense primer SEQ ID No: 16 or SEQ ID No: 17 or SEQ ID No: 26 or SEQ ID No: 27,
a pair of primers 2 targeting the variable regions V4-V6 of the gene 16S made up:
   of a sense primer of SEQ ID No: 28 or SEQ ID No: 29 or SEQ ID No: 30, and
   of an antisense primer SEQ ID No: 31 or SEQ ID No: 32 or SEQ ID No: 33,
a pair of primers 3 targeting the variable regions V7-V9 of the gene 16S made up:
   of a sense primer of SEQ ID No: 34 or SEQ ID No: 35, and
   of an antisense primer SEQ ID No: 36 or SEQ ID No: 37 or SEQ ID No: 38.

The kit according to the invention is preferably capable of implementing the method according to the invention.

The invention also relates to the use of the kit according to the invention for detecting the microorganisms present in the case of infectious diseases, such as bacteria, viruses or yeasts. The invention preferably relates to the use of the kit for diagnosing an infectious disease in a subject or a plant.

The invention also relates to the use of the kit according to the invention for detecting at least one genetic disease or at least one cancer.

### EXAMPLE

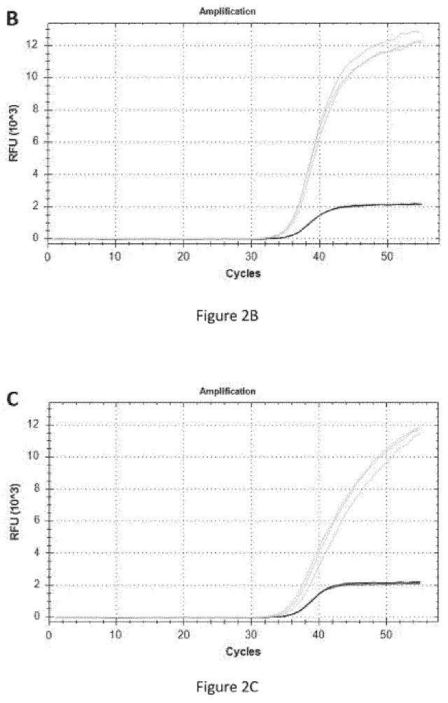

### Example 1 of the Method According to the Invention for the Amplification of Two Sequences of a Specific Gene of Staphylococcus aureus and of Escherichia coli (FIG. 2A/2B/2C)

Materials and Method

The Relay-PCR reactions were carried out in the following manner: 0.2 mM of dNTP, 4 mM of MgCl2, 0.05 of a unit of FastStart polymerase (Roche) and the buffer according to the supplier's conditions. The TaqMan probes for the pair of primers 1 were at 0.25 mM, while those for the pair of primers 2 were at 0.1 mM. The pairs of primers were set at different concentrations: 1 mM for the standard Duplex, 1 mM for the pair of primers 1 in the Duplex-Relays and 0.1 mM or 1 mM for the pair of primers 2 in the Duplex-Relays. The cycles were as follows: denaturation at 95° C. for 10 minutes, then 55 cycles at 95° C. 15 sec/55° C. 15 sec/68° C. 15 sec. Approximately 100 equivalent genome copies of each of the bacteria tested were added to the mix; in order to perform the amplifications.

Results

The amplification of the two sequences of interest was performed by a standard Duplex-PCR; by Relay-PCR according to the invention, having a ratio of pair of primers 1:pair of primers 2 of 10:1, and by Relay-PCR according to the invention, having a ratio of pair of primers 1:pair of primers 2 of 1:1.

Within the context of the standard Duplex-PCR, the two pairs of primers were used at a high concentration. The pair of primers making it possible to amplify the specific sequence of the gene Staphylococcus aureus is formed of primers of sequence SEQ ID No: 3 and SEQ ID No: 4, and the pair making it possible to amplify Escherichia co/i is made up of primers of sequence SEQ ID No: 1 and SEQ ID No: 2. The results are set out in FIG. 2A and Table 3. The inventors have thus observed a superimposition of the Cq (number of cycles initiating a detection of the fluorescence signal), meaning that an identical quantity of bacterial genome has indeed been added to the PCR mix. Moreover; just as much of a quantity of amplicons of Escherichia coli and Staphylococcus aureus were generated.

During the Duplex-Relay PCR having a ratio of 10:1, the pair of primers 1 is formed of primers of sequences SEQ ID No: 2 and SEQ ID No: 3, and the pair of primers 2 is made up of primers of sequences SEQ ID No: 7 and SEQ ID No: 8. The first pair of primers makes it possible to amplify the specific sequence of the gene Staphylococcus aureus, and the second pair of hybrid or chimeric primers makes it possible to amplify the specific sequence of the gene of Escherichia coli. The results are set out in FIG. 2B and Table 3. The inventors have thus observed a Cq offset of the curves between the Staphylococcus aureus and Escherichia coli amplicons. This delay brought about by the Duplex-Relay PCR was of 2.09 cycles (35.27-33.18) for a primer ratio of 10:1, i.e. approximately a factor 4 (2\hat{ }1.99). It leads to a differential amount of approximately 4 times fewer amplicons relating to the gene Escherichia coli compared with amplicons of Staphylococcus aureus.

During the Relay-PCR having a ratio 1:1, the pairs of primers used are identical to those used in the Relay-PCR (ratio 10:1). The results are set out in FIG. 2C and Table 3. The inventors also observed a Cq offset of the curves between the Staphylococcus aureus and Escherichia coli amplicons. This delay brought about by the Duplex-Relay PCR was of 1.79 cycles (35.68-33.89), i.e. approximately a factor 3.5 (2\hat{ }1.79). The differential amount of amplicons was observed here too, for similar factors.

TABLE 3

| | | M. | SD. | | | |
|---|---|---|---|---|---|---|
| | Cq SA | Cq SA | Cq SA | Cq EC | M. EC | SD. EC |
| Duplex- | 32.61 | 32.95 | 0.31 | 32.99 | 32.90 | 0.11 |
| PCR | 33.04 | | | 32.93 | | |
| | 33.2 | | | 32.78 | | |
| Relay- | 33.06 | 33.18 | 0.20 | 35.37 | 35.27 | 0.10 |
| PCR | 33.41 | | | 35.28 | | |
| 10:1 | 33.08 | | | 35.17 | | |
| Relay- | 34.42 | 33.89 | 0.51 | 35.77 | 35.68 | 0.08 |
| PCR | 33.85 | | | 35.62 | | |
| 1:1 | 33.4 | | | 35.65 | | |

M. Cq = mean of the Cq.
SD. Cq = standard deviation of Cq.
SA: values for Staphylococcus aureus
EC: values for Escherichia coli.

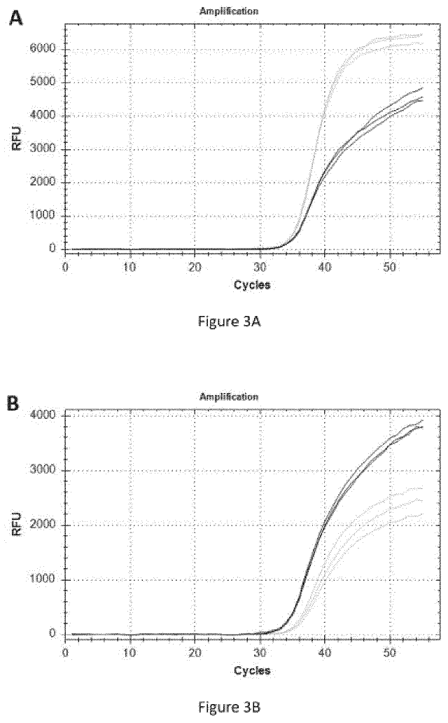

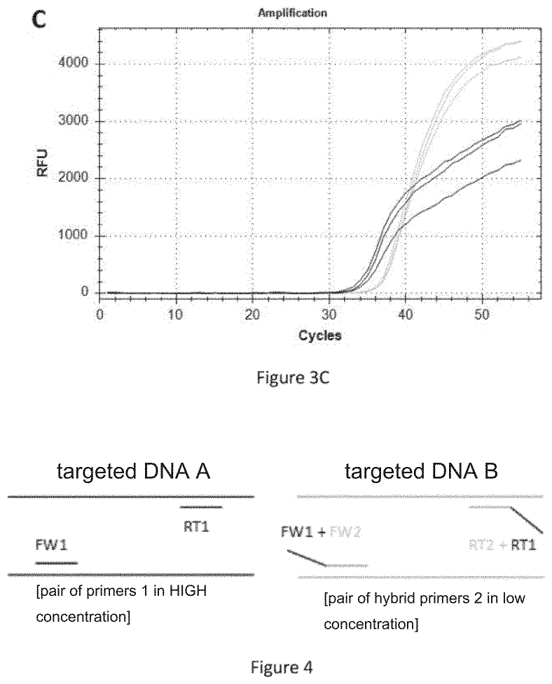

### Example 2 of the Method According to the Invention for the Amplification of Two Sequences of a Specific Gene of Klebsiella pneumoniae and Staphylococcus aureus (FIG. 3A/3B/3C)

The material and method are identical to that described in Example 1.

Results

The amplification of the two sequences of interest was performed by a standard Duplex-PCR, by Duplex-Relay PCR according to the invention, having a ratio of pair of primers 1:pair of primers 2 of 10:1, and by Duplex-Relay PCR according to the invention, having a ratio of pair of primers 1:pair of primers 2 of 1:1.

Within the context of the standard Duplex-PCR, the two pairs of primers were used at a high concentration. The pair of primers making it possible to amplify the specific sequence of the gene Klebsiella pneumoniae is formed of primers of sequence SEQ ID No: 5 and SEQ ID No: 6, and the pair making it possible to amplify Staphylococcus aureus is made up of primers of sequences SEQ ID No: 3 and SEQ ID No: 4. The results are set out in FIG. 3A and Table 4. The inventors have thus observed a superimposition of the Cq (number of cycles initiating a detection of the fluorescence signal), meaning that an identical quantity of bacterial genome has indeed been added to the PCR mix. Moreover, just as much of a quantity of amplicons of Klebsiella pneumoniae and Staphylococcus aureus were generated.

During the Relay-PCR having a ratio of 10:1, the pair of primers 1 is formed of primers of sequences SEQ ID No: 5 and SEQ ID No: 6, and the pair of primers 2 is made up of primers of sequences SEQ ID No: 9 and SEQ ID No: 10. The first pair of primers makes it possible to amplify the specific sequence of the gene Klebsiella pneumoniae, and the second pair of chimeric primers makes it possible to amplify the specific sequence of the gene Staphylococcus aureus. The results are set out in FIG. 3B and Table 4. The inventors have thus observed a Cq offset of the curves between the Klebsiella pneumoniae and Staphylococcus aureus amplicons. This delay brought about by the Duplex-Relay PCR was of 1.99 cycles (35.35-33.36) for the primer ratio of 10:1, i.e. approximately a factor 4 (2\hat{ }1.99). It leads to a differential amount of approximately 4 times fewer amplicons relating to the gene Staphylococcus aureus compared with amplicons of Klebsiella pneumoniae.

During the Relay-PCR having a ratio 1:1, the pairs of primers used are identical to those used in the Relay-PCR (ratio 10:1). The results are set out in FIG. 3C and Table 4. The inventors have also observed a Cq offset of the curves between the Klebsiella pneumoniae and Staphylococcus aureus amplicons. The delay brought about by the Duplex-Relay PCR was of 2.59 cycles (36.43-33.84), i.e. approximately a factor 6 (2\hat{ }2.59). It leads to a differential amount of approximately 6 times fewer amplicons relating to the gene Staphylococcus aureus compared with amplicons of Klebsiella pneumoniae.

TABLE 4

| | | M. | SD. | | | |
|---|---|---|---|---|---|---|
| | Cq KP | Cq KP | Cq KP | Cq SA | M. SA | SD. SA |
| Duplex- | 33.9 | 33.85 | 0.10 | 33.15 | 33.44 | 0.08 |
| PCR | 33.91 | | | 33.47 | | |
| | 33.73 | | | 33.35 | | |
| Relay- | 33.5 | 33.36 | 0.15 | 35.09 | 35.35 | 0.27 |
| PCR | 33.37 | | | 35.63 | | |
| 10:1 | 33.2 | | | 35.32 | | |
| Relay- | 33.31 | 33.84 | 0.51 | 36.27 | 36.43 | 0.15 |
| PCR | 33.89 | | | 36.45 | | |
| 1:1 | 34.33 | | | 36.56 | | |

M. Cq = mean of the Cq.
SD. Cq = standard deviation of Cq.
KP: values for Klebsiella pneumoniae
SA: values for Staphylococcus aureus

### Example 3 of the Method According to the Invention Compared with Standard Multiplex PCR for the Amplification of Two Sequences of a Gene 16S of Klebsiella pneumoniae (FIG. 6)

Material and Method

The Relay-PCR reactions were carried out in the following manner: 0.2 mM of dNTP, 4 mM of MgCl2, 0.05 of a unit of FastStart polymerase (Roche) and the buffer according to the supplier's conditions. The pairs of primers were set at different concentrations: 1 mM for the standard simplex, 1 mM for the standard Duplex, 1 mM for the pair of primers 1 in the Duplex-Relays and 0.04 mM for the pair of primers 2 in the Duplex-Relays. The cycles were as follows: denaturation at 95° C. for 10 minutes, then 65 cycles at 95° C. 15 sec/55° C. 15 sec/68° C. 30 sec. Approximately 100 equivalent genome copies of Klebsiella pneumoniae were added to the mix, in order to perform the amplifications.

Results

The amplification of the two sequences of interest was performed by a standard Duplex-PCR (FIG. 6, line 3), and by Duplex-Relay PCR according to the invention (FIG. 6, line 4), having a ratio of pair of primers 1:pair of primers 2 of 25:1.

Line 1 represents the amplicon of the region 5′ of the gene 16S (covering the variable zones V1 to V2) (control PCR).

Line 2 represents the amplicon of the central region of the gene 16S (covering the variable zones V4 to V6) (control PCR).

Line 3 represents the amplicons in standard multiplex PCR of the regions V1-V2 and V4-V6 of the gene 16S.

Line 4 represents the amplicons of the regions V1-V2 and V4-V6 of the gene 16S, with the method according to the present invention (multiplex Relay-PCR).

Within the context of the standard Duplex-PCR, the two pairs of primers were used at a high concentration (Line 3). The pair of primers making it possible to amplify the sequence V1V2 of the gene 16S of Klebsiella pneumoniae is formed of primers of sequence SEQ ID No: 14 and SEQ ID No: 16, and the pair making it possible to amplify the sequence V4-V6 of the gene 16S of Klebsiella pneumoniae is made up of primers of sequences SEQ ID No: 20 and SEQ ID No: 23. The results are set out in FIG. 6. The inventors have observed (line 3 and left-hand arrow) that there is no amplicon of the region V1-V2 of the gene 16S, demonstrating that the standard conditions of multiplex PCR are not compatible with the simultaneous amplification of 2 regions of the gene 16S.

During the Relay-PCR having a ratio of 25:1, the pair of primers 1 is formed of primers of sequences SEQ ID No: 14 and SEQ ID No: 16, and the pair of primers 2 is made up of primers of sequences SEQ ID No: 28 and SEQ ID No: 33. The first pair of primers makes it possible to amplify the region V1-V2 of the gene 16S of Klebsiella pneumoniae, and the second pair of chimeric primers makes it possible to amplify the region V4-V6 of the gene 16S of Klebsiella pneumoniae. The results are set out in FIG. 6. The inventors have thus observed that the conditions of the Relay-PCR make it possible to simultaneously co-amplify the 2 regions of the gene 16S (Line 4), and that the amplicons of the region V1-V2 are present in a larger quantity than those of the central region (more intense signal for the amplicon V1-V2, even though it is smaller in size).

Consequently, the method according to the present invention makes it possible to co-amplify the two targeted regions, contrary to a standard multiplex PCR. The particular structure of the chimeric primers according to the present invention thus makes it possible to overcome the problems of the prior art, in particular of multiplex PCR.

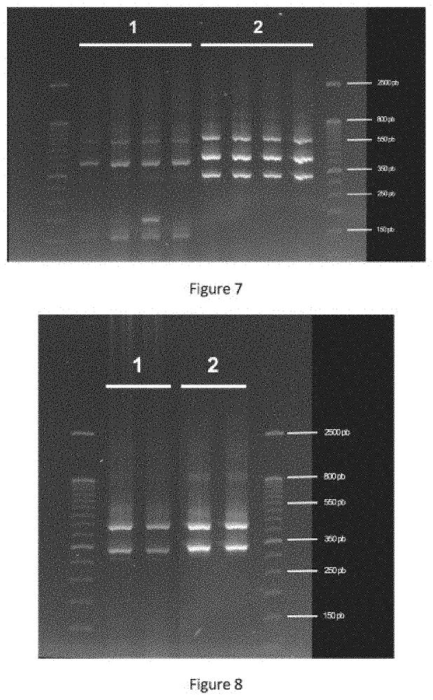

### Example 4 of the Method According to the Invention Compared with Standard Multiplex PCR for the Amplification of Three Sequences of the Gene 16S of Klebsiella pneumoniae (FIG. 7)

Material and Method

The Relay-PCR reactions were carried out in the following manner: 0.2 mM of dNTP, 4 mM of MgCl2, 0.05 of a unit of FastStart polymerase (Roche) and the buffer according to the supplier's conditions. The pairs of primers were set at different concentrations: 1 mM for the standard Triplex, 1 mM for the pair of primers 1 in the Triplex-Relays and 0.04 mM for the pair of primers land the pairs of primers 3 in the Triplex-Relays. The cycles were as follows: denaturation at 95° C. for 10 minutes, then 65 cycles at 95° C. 15 sec/55° C. 15 sec/68° C. 18 sec. Approximately 100 equivalent genome copies of Klebsiella pneumoniae were added to the mix, in order to perform the amplifications,

Results

Within the context of the standard Triplex-PCR, the three pairs of primers were used at a high concentration (Line 1). The pair of primers making it possible to amplify the specific sequence (V1-V2) of the gene 16S of Klebsiella pneumoniae is formed of primers of sequence SEQ ID No: 14 and SEQ ID No: 16, the pair making it possible to amplify the specific sequence (V4-V6) of the gene 16S of Klebsiella pneumoniae is made up of primers of sequences SEQ ID No: 20 and SEQ ID No: 23, and the pair making it possible to amplify the specific sequence (V7-V9) of the gene 16S of Klebsiella pneumoniae is made up of primers of sequences SEQ ID No: 24 and SEQ ID No: 25.

Within the context of the Triplex-Relay PCR (Line 2), the pair of primers making it possible to amplify the specific sequence (V1V2) of the gene 16S of Kiebsiella pneumoniae is formed of primers of sequence SEQ ID No: 14 and SEQ ID No: 16, the pair making it possible to amplify the specific sequence (V4-V6) of the gene 16S of Klebsiella pneumoniae is made up of primers of sequences SEQ ID No: 28 and SEQ ID No: 33, and the pair making it possible to amplify the specific sequence (V7-V9) of the gene 16S of Kiebsiella pneumoniae is made up of primers of sequences SEQ ID No: 34 and SEQ ID No: 36. The primers of the pair targeting the region V1-V2 are set at a high concentration, while the hybrid or chimeric primers of the other pairs are set at a low concentration. The results are set out in FIG. 7.

The inventors have observed that the targeted regions were poorly amplified by the standard Triplex, in particular the variable regions V1 and V2 of the gene 16S of Kiebsiella pneumoniae. In contrast, the Triplex-Relay is capable of amplifying the 3 regions of the gene 16S of Klebsiella pneumoniae, i.e. the region V1 to V2 (343 bp), the region V7 to V9 (413 bp in standard Duplex-PCR and 447 bp in Duplex-Relay PCR), and the region V4 to V6 (509 bp in standard Duplex-PCR and 543 bp in Duplex-Relay PCR).

Consequently, the method according to the present invention makes it possible to co-amplify the three targeted regions.

### Example 5 of the Method According to the Invention, with a Pair of Primers 1 Comprising One Primer in a High Concentration, and One Hybrid or Chimeric Primer in a Low Concentration (FIG. 8)

Material and Method

The two Relay-PCR reactions were carried out in the following manner: 0.2 mM of dNTP, 4 mM of MgCl2, 0.05 of a unit of FastStart polymerase (Roche) and the buffer according to the supplier's conditions. Under the first condition of Duplex-Relay, the primers were set to different concentrations: 1 mM for the two primers of the pair 1 and 0.04 mM for the pair of primers 2. Under the second condition of Duplex-Relay, the primers were set to different concentrations: 1 mM for the first standard primer of the pair 1, 0.04 mM for the second chimeric primer of the pair 1, and 0.04 mM for the chimeric primers of the pair 2. The cycles were as follows: denaturation at 95° C. for 10 minutes, then 55 cycles at 95° C. 15 sec/55° C. 15 sec/68° C. 45 sec. Approximately 100 equivalent genome copies of Kiebsiella pneumoniae were added to the mix, in order to perform the amplifications.

Results

Within the context of the first Duplex-Relay PCR (Line 1), the pair of primers making it possible to amplify the sequence V1-V2 of the gene 16S of Kiebsiella pneumoniae is formed of primers of sequence SEQ ID No: 14 and SEQ ID No: 16 at a high concentration, the pair making it possible to amplify the region V7-V9 of the gene 16S of Kiebsiella pneumoniae is made up of chimeric primers of sequences SEQ ID No: 34 and SEQ ID No: 36 at a low concentration.

Within the context of the second Duplex-Relay PCR (Line 2), one single primer of the pair 1 was set at a high concentration, SEQ ID No: 14, the other primer being a hybrid primer set at a low concentration, SEQ ID No: 26. The pair of primers making it possible to amplify the region V1V2 of the gene 16S of Kiebsiella pneumoniae is formed of primers of sequence SEQ ID No: 14 and SEQ ID No: 26, the pair making it possible to amplify the region V7-V9 of the gene 16S of Kiebsiella pneumoniae is made up of primers of sequences SEQ ID No: 34 and SEQ ID No: 37. The results are set out in FIG. 8.

The inventors have observed that, under the 2 conditions, the amplicons corresponding to the regions V1-V2 (343 bp for the first Duplex-Relay PCR (line 1) and 349 bp for the second Duplex-Relay PCR (line 2)), and V7-V9 (447 bp for the first Duplex-Relay PCR (line 1) and 453 bp for the second Duplex-Relay PCR (line 2) are successfully amplified. However, it is noted that the fact of using a single primer of the pair 1 at a high concentration appears to even further facilitate the Relay-PCR according to the present invention. Moreover, the brightness of the strip of amplicons V1-V2 identical to that of the strip V7-V9 suggests that the quantity of amplicon V1-V2 is much greater than that of the amplicons V7-V9, the amplicons V1-V2 being smaller. This variation of the method of the invention thus preserves the favored amplification of the region for which a primer at a high concentration is used.

1. A method for amplifying at least two distinct nucleic acid sequences of interest, present in a sample, using a pair of primers 1 capable of amplifying a first nucleic acid sequence of interest (A), and at least one pair of primers capable of amplifying a second nucleic acid sequence of interest (B), said method comprising the following steps:
a. contacting the sample with the pair of primers 1 capable of amplifying the nucleic acid sequence (A) and the pair of primers 2 capable of amplifying the second nucleic acid sequence (B),
i. the pair of primers 1 being formed of a first primer and a second primer which are capable of amplifying a nucleic acid corresponding to the first sequence (A),
ii. the pair of primers 2 being formed of a first primer and a second primer which are capable of amplifying a nucleic acid corresponding to the second sequence (B),
b. adding selected reagents, and conditions allowing for PCR amplification, and
c. amplifying the first and second nucleic acid sequences (A and B),
characterized in that the sequence of the first primer of the pair of primers 2 further comprises, at 5′, the sequence of the first or of the second primer of the pair of primers 1, and the sequence of the second primer of the pair of primers 2 further comprises, at 5′, the sequence of the first or of the second primer of the pair of primers 1.

2. The method according to claim 1, characterized in that the sequence of the first primer of the pair of primers 2 and the sequence of the first or of the second primer of the pair of primers 1 are adjacent, and the sequence of the second primer of the pair of primers 2 and the sequence of the first or of the second primer of the pair of primers 1 are adjacent.

3. The method according to claim 1, characterized in that the second primer of the pair 1 is a hybrid primer comprising, at 5′, the sequence of the first primer of the pair 1, and said sequences are adjacent.

4. The method according to claim 1, characterized in that the concentration of the pair of primers 2 is less than or equal to that of the pair of primers 1.

5. The method according to claim 4, characterized in that the concentration ratio between the pair of primers 1 and the pair of primers 2 is between 1:1 and 1000:1.

6. The method according to claim 5, characterized in that the concentration ratio between the pair of primers 1 and the pair of primers 2 is between 1:1 and 100:1, preferably 25:1.

7. The method according to claim 1, further comprising contacting the sample with a pair of primers 3 capable of amplifying a nucleic acid sequence corresponding to the 3rd nucleic acid sequence (C), characterized in that the sequence of the first primer of the pair of primers 3 further comprises, at 5′, the sequence of the first or of the second primer of the pair of primers 1, and the sequence of the second primer of the pair of primers 3 further comprises, at 5′, the sequence of the first or of the second primer of the pair of primers 1.

8. The method according to claim 7, wherein the concentration of the pair of primers 1 is greater than that of the concentration of the second and/or third pair.

9. The method according to claim 8, wherein the concentration of the pair of primers 2 is equal to that of the pair of primers 3.

10. The method according to claim 7, further comprising contacting the sample with a pair of primers 4 capable of amplifying a 4th nucleic acid sequence (D), characterized in that the sequence of the first primer of the pair of primers 4 further comprises, at 5′, the sequence of the first or of the second primer of the pair of primers 1, and the sequence of the second primer of the pair of primers 4 further comprises, at 5′, the sequence of the first or of the second primer of the pair of primers 1.

11. The method according to claim 10, wherein the concentration of the pair of primers 4 is less than that of the pair of primers 1.

12. The method according to claim 11, wherein the concentration of the pair of primers 2, of the pair of primers 3 and of the pair of primers 4 is identical.

13. The method according to claim 1, further comprising a step of sequencing the amplified nucleic acids.

14. The method according to claim 1, wherein the amplification of nucleic acid sequences of interest makes it possible to detect and identify at least one microorganism involved in at least one infectious disease.

15. The method according to claim 14, characterized in that the microorganisms are identified by fluorescent probes, fluorescent marking, melting curve, nested PCR, quantitative PCR, reverse transcription PCR, or by DNA sequencing.

16. A kit comprising:
a pair of primers 1 targeting the variable regions V1-V2 of the gene 16S made up:
of a sense primer of SEQ ID No: 14 or SEQ ID No: 15, and
of an antisense primer SEQ ID No: 16 or SEQ ID No: 17 or SEQ ID No: 26 or SEQ ID No: 27,
a pair of primers 2 targeting the variable regions V4-V6 of the gene 16S made up:
of a sense primer of SEQ ID No: 28 or SEQ ID No: 29 or SEQ ID No: 30, and
of an antisense primer SEQ ID No: 31 or SEQ ID No: 32 or SEQ ID No: 33.

17. A kit comprising:
a pair of primers 1 targeting the variable regions V1-V2 of the gene 16S made up:
of a sense primer of SEQ ID No: 14 or SEQ ID No: 15, and
of an antisense primer SEQ ID No: 16 or SEQ ID No: 17 or SEQ ID No: 26 or SEQ ID No: 27,
a pair of primers 2 targeting the variable regions V7-V9 of the gene 16S made up:
of a sense primer of SEQ ID No: 34 or SEQ ID No: 35, and
of an antisense primer SEQ ID No: 36 or SEQ ID No: 37 or SEQ ID No: 38.

18. A kit comprising:
a pair of primers 1 targeting the variable regions V1-V2 of the gene 16S made up:
of a sense primer of SEQ ID No: 14 or SEQ ID No: 15, and
of an antisense primer SEQ ID No: 16 or SEQ ID No: 17 or SEQ ID No: 26 or SEQ ID No: 27,
a pair of primers 2 targeting the variable regions V4-V6 of the gene 16S made up:
of a sense primer of SEQ ID No: 28 or SEQ ID No: 29 or SEQ ID No: 30, and
of an antisense primer SEQ ID No: 31 or SEQ ID No: 32 or SEQ ID No: 33,
a pair of primers 3 targeting the variable regions V7-V9 of the gene 16S made up:
of a sense primer of SEQ ID No: 34 or SEQ ID No: 35, and
of an antisense primer SEQ ID No: 36 or SEQ ID No: 37 or SEQ ID No: 38.

19. Use of a kit according to claim 16 in a method for detecting at least one microorganism present in the case of at least one infectious disease.


SEQUENCE LISTING (38 sequences)

SEQ ID NO: 1 - DNA, 20 bp, Artificial Sequence
  1st pair
cggaagcaacgcgtaaactc

SEQ ID NO: 2 - DNA, 21 bp, Artificial Sequence
  1st pair
tgagcgtcgcagaacattaca

SEQ ID NO: 3 - DNA, 26 bp, Artificial Sequence
  1st pair
tcgaaattaaatgttgtcgtgtcttc

SEQ ID NO: 4 - DNA, 34 bp, Artificial Sequence
  1st pair
caatattttcatttttgacatggagagaaacatc

SEQ ID NO: 5 - DNA, 22 bp, Artificial Sequence
  1st pair
gacgggatatctgaccagtcgg

SEQ ID NO: 6 - DNA, 22 bp, Artificial Sequence
  1st pair
ccgggttttgcgtaatgatctg

SEQ ID NO: 7 - DNA, 46 bp, Artificial Sequence
  2nd pair
tcgaaattaaatgttgtcgtgtcttccggaagcaacgcgtaaactc

SEQ ID NO: 8 - DNA, 55 bp, Artificial Sequence
  2nd pair
caatattttcatttttgacatggagagaaacatctgagcgtcgcagaacattaca

SEQ ID NO: 9 - DNA, 48 bp, Artificial Sequence
  2nd pair
gacgggatatctgaccagtcggtcgaaattaaatgttgtcgtgtcttc

SEQ ID NO: 10 - DNA, 56 bp, Artificial Sequence
  2nd pair
ccgggttttgcgtaatgatctgcaatattttcatttttgacatggagagaaacatc

SEQ ID NO: 11 - DNA, 20 bp, Artificial Sequence
  Probe 1 - EC
cgcgtccgatcacctgcgtc

SEQ ID NO: 12 - DNA, 22 bp, Artificial Sequence
  Probe 2 - KP
gttgaggtcaacagtgctgcgg

SEQ ID NO: 13 - DNA, 21 bp, Artificial Sequence
  Probe 3 - SA
tcgcgacattcattatgccca

SEQ ID NO: 14 - DNA, 20 bp, Artificial Sequence
  V1sense
agagtttgatcctggctcag

SEQ ID NO: 15 - DNA, 17 bp, Artificial Sequence
  V1sense
gcctaacacatgcaagt

SEQ ID NO: 16 - DNA, 14 bp, Artificial Sequence
  V2antisense
gcctcccgtaggag

SEQ ID NO: 17 - DNA, 18 bp, Artificial Sequence
  V2antisense
tctggaccgtgtctcagt

SEQ ID NO: 18 - DNA, 14 bp, Artificial Sequence
  V3sense
ctcctacgggaggc

SEQ ID NO: 19 - DNA, 12 bp, Artificial Sequence
  V3antisense
accgcggctgct

SEQ ID NO: 20 - DNA, 18 bp, Artificial Sequence
  V4sense
cgtgccagcagccgcggt

SEQ ID NO: 21 - DNA, 19 bp, Artificial Sequence
  V4sense
cggaattactgggcgtaaa

SEQ ID NO: 22 - DNA, 15 bp, Artificial Sequence
  V6antisense
acacgagctgacgac

SEQ ID NO: 23 - DNA, 20 bp, Artificial Sequence
  V6antisense
tcacgacacgagctgacgac

SEQ ID NO: 24 - DNA, 14 bp, Artificial Sequence
  V7sense
aacgagcgcaaccc

SEQ ID NO: 25 - DNA, 22 bp, Artificial Sequence
  V9antisense
tacggttaccttgttacgactt

SEQ ID NO: 26 - DNA, 38 bp, Artificial Sequence
  V1sense-V2antisense
agagtttgatcctggctcagtctggaccgtgtctcagt

SEQ ID NO: 27 - DNA, 35 bp, Artificial Sequence
  V1sense-V2antisense
gcctaacacatgcaagttctggaccgtgtctcagt

SEQ ID NO: 28 - DNA, 38 bp, Artificial Sequence
  V1sense-V4sense
agagtttgatcctggctcagcgtgccagcagccgcggt

SEQ ID NO: 29 - DNA, 39 bp, Artificial Sequence
  V1sense-V4sense
agagtttgatcctggctcagcggaattactgggcgtaaa

SEQ ID NO: 30 - DNA, 36 bp, Artificial Sequence
  V1sense-V4sense
gcctaacacatgcaagtcggaattactgggcgtaaa

SEQ ID NO: 31 - DNA, 35 bp, Artificial Sequence
  V1sense-V6antisense
agagtttgatcctggctcagacacgagctgacgac

SEQ ID NO: 32 - DNA, 32 bp, Artificial Sequence
  V1sense-V6antisense
gcctaacacatgcaagtacacgagctgacgac

SEQ ID NO: 33 - DNA, 34 bp, Artificial Sequence
  V2antisense-V6antisense
gcctcccgtaggagtcacgacacgagctgacgac

SEQ ID NO: 34 - DNA, 34 bp, Artificial Sequence
  V1sense-V7sense
agagtttgatcctggctcagaacgagcgcaaccc

SEQ ID NO: 35 - DNA, 32 bp, Artificial Sequence
  V2antisense-V7sense
tctggaccgtgtctcagtaacgagcgcaaccc

SEQ ID NO: 36 - DNA, 36 bp, Artificial Sequence
  V2antisense-V9antisense
gcctcccgtaggagtacggttaccttgttacgactt

SEQ ID NO: 37 - DNA, 42 bp, Artificial Sequence
  V1sense-V9antisense
agagtttgatcctggctcagtacggttaccttgttacgactt

SEQ ID NO: 38 - DNA, 39 bp, Artificial Sequence
  V1sense-V9antisense
gcctaacacatgcaagttacggttaccttgttacgactt